(19) United States
(12) Patent Application Publication
(10) Pub. No.: US 2022/0189032 A1
(43) Pub. Date: Jun. 16, 2022

(54) CEREBRAL STROKE EARLY ASSESSMENT METHOD AND SYSTEM, AND BRAIN REGION SEGMENTATION METHOD

(71) Applicant: GE Precision Healthcare LLC, Wauwatosa, WI (US)

(72) Inventors: Linshang Rao, Guangzhou (CN); Ling Liu, Shanghai (CN); Chen Zhang, Guangzhou (CN); Zhoushe Zhao, Beijing (CN)

(21) Appl. No.: 17/550,406

(22) Filed: Dec. 14, 2021

(30) Foreign Application Priority Data
Dec. 15, 2020 (CN) 202011492893.8

(51) Int. Cl. G06T 7/11

(52) U.S. Cl. CPC G06T 7/11; G06T 2207/30016; G06T 2207/20081; G06T 2207/20084; G16H 30/20; G06T 2207/10108; G06T 2207/10088; G06T 2207/10081; G06T 2207/10104

(57) ABSTRACT

A cerebral stroke early assessment system for cerebral stroke early assessment, comprising a preprocessing module, configured to preprocess an acquired brain medical image set; a brain partitioning module, configured to perform brain region segmentation on the preprocessed brain medical image set, the brain partitioning module comprising an image segmentation neural network and the image segmentation neural network being trained with the aid of an auto-encoder; and a scoring module, configured to perform scoring on the basis of a brain partition image obtained by the brain partitioning module. The present disclosure can improve the segmentation accuracy of brain partition images and the accuracy of cerebral stroke early assessment.

### CROSS-REFERENCE TO RELATED APPLICATIONS

This application claims priority to Chinese Patent Application No. 202011492893.8, filed on Dec. 15, 2020, the disclosure of which is incorporated herein by reference in its entirety.

### BACKGROUND

The present disclosure relates to medical imaging, and in particular to a cerebral stroke early assessment method and system, and a brain region segmentation method.

Cerebral stroke is currently one of major diseases that leads to death, wherein acute ischemic stroke is a brain function impairment resulting from a loss of blood supply to brain tissues due to various causes, and is the major type of cerebral stroke, accounting for about 60% to 80% of all stroke types. Positive treatment of patients with brain ischemia at an early stage can prevent further development of the brain ischemia, and can mitigate brain damage and avoid possible death caused by irreversible necrosis of brain tissues. Cerebral stroke early assessment methods in clinical application can employ ASPECTS (Alberta Stroke Program Early CT Score) scoring, which provides physicians with quantified disease information to help further formulate effective treatment regimens. ASPECTS divides the important levels of the middle cerebral artery blood supply into ten regions according to cranial CT image data or other modality image data, comprising caudate nucleus (C), lenticular nucleus (L), posterior limb of internal capsule (IC), insular zone (I), M1 (middle cerebral artery anterior cortex), M2 (middle cerebral artery lateral insular cortex), and M3 (middle cerebral artery posterior cortex) located at a nucleus level, and M4 (middle cerebral artery cortex above M1), M5 (middle cerebral artery cortex above M2), and M6 (middle cerebral artery cortex above M3) located at a level above the nucleus (supranuclear layer). The above ten regions have the same weights, each having a score of 1, and a total score of 10. The number of regions where early ischemic changes occur is subtracted from the total score, and the resultant value is used as a scoring result to provide a basis for condition evaluation and treatment.

A method for ASPECTS scoring is based on a judgment made by a physician with just the naked eye; however, due to the existence of factors such as different imaging apparatuses, different technicians, and different patient conditions, the consistency of cranial CT image data cannot be ensured, and the subjectivity results in large differences. Another method for ASPECTS scoring is template alignment-based, in which an acquired brain CT image is aligned with a corresponding ASPECTS brain partition template image, and individual partitions marked in the ASPECTS brain partition template image are mapped to the brain CT image aligned therewith, so as to obtain a plurality of ASPECTS brain partitions in the brain CT image. The ASPECTS scoring method based on template alignment suffers from shortcomings such as: (1) when an alignment algorithm searches for two similar regions, matching misalignment will be caused if the source image has excessive noise; (2) the difference in image data distribution between different apparatuses is large, and the template-based alignment method is difficult to apply to data derived from different apparatuses; and (3) for cases in which a brain structure greatly differs from a standard template structure, the template-based alignment algorithm has difficulty reaching an accurate score.

Therefore, there is a need for a cerebral stroke early assessment method and system, and a corresponding brain region segmentation method, capable of reducing subjective differences introduced by medical personnel based on empirical judgment and improving accuracy.

### SUMMARY

In one aspect of the invention, provided is a medical image-based cerebral stroke early assessment system, comprising: a preprocessing module, configured to preprocess an acquired brain medical image; a brain partitioning module, configured to perform brain region segmentation on the preprocessed brain medical image, the brain partitioning module comprising an image segmentation neural network and the image segmentation neural network being trained with the aid of an auto-encoder; and a scoring module, configured to perform scoring on the basis of a brain partition image obtained by the brain partitioning module.

In the aspect of the present disclosure, in the cerebral stroke early assessment system, the image segmentation neural network includes a Dense V-Net neural network, a U-Net neural network, or a V-Net neural network.

In the aspect of the present disclosure, in the cerebral stroke early assessment system, the auto-encoder includes a variational auto-encoder.

In the aspect of the present disclosure, in the cerebral stroke early assessment system, the auto-encoder is connected to a down-sampling branch of the image segmentation neural network.

In the aspect of the present disclosure, in the cerebral stroke early assessment system, a loss function trained by the image segmentation neural network includes a KL divergence loss function and a Dice coefficient loss function, wherein the KL divergence loss function corresponds to a loss function of the auto-encoder, and the Dice coefficient loss function corresponds to a loss function of the image segmentation neural network.

In another aspect of the present disclosure, provided is a medical image-based cerebral stroke early assessment method, comprising: preprocessing an acquired brain medical image; performing brain region segmentation on the preprocessed brain medical image, the brain region segmentation using an image segmentation neural network and the image segmentation neural network being trained with the aid of an auto-encoder; and performing scoring on the basis of a brain partition image obtained by the brain partitioning module.

In the aspect of the present disclosure, in the cerebral stroke early assessment method, the image segmentation neural network includes a Dense V-Net neural network, a U-Net neural network, or a V-Net neural network.

In the aspect of the present disclosure, in the cerebral stroke early assessment method, the auto-encoder includes a variational auto-encoder.

In the aspect of the present disclosure, in the cerebral stroke early assessment method, the auto-encoder is connected to a down-sampling branch of the image segmentation neural network.

In the aspect of the present disclosure, in the cerebral stroke early assessment method, a loss function trained by the image segmentation neural network includes a KL divergence loss function and a Dice coefficient loss function, wherein the KL divergence loss function corresponds to a loss function of the auto-encoder, and the Dice coefficient loss function corresponds to a loss function of the image segmentation neural network.

In yet another aspect of the present disclosure, provided is a brain region segmentation method for a brain medical image, comprising: preprocessing an acquired brain medical image; and performing brain region segmentation on the preprocessed brain medical image by using an image segmentation neural network, the image segmentation neural network being trained with the aid of an auto-encoder.

In the aspect of the present disclosure, in the brain region segmentation method, the image segmentation neural network includes a Dense V-Net neural network, a U-Net neural network, or a V-Net neural network.

In the aspect of the present disclosure, in the brain region segmentation method, the auto-encoder includes a variational auto-encoder.

In the aspect of the present disclosure, in the brain region segmentation method, the auto-encoder is connected to a down-sampling branch of the image segmentation neural network.

In the aspect of the present disclosure, in the brain region segmentation method, a loss function trained by the image segmentation neural network includes a KL divergence loss function and a Dice coefficient loss function, wherein the KL divergence corresponds to a loss function of the auto-encoder, and the Dice coefficient loss function corresponds to a loss function of the image segmentation neural network.

In the aspect of the present disclosure, provided is a system, comprising a processor configured to perform the foregoing cerebral stroke early assessment method and brain region segmentation method.

Provided is a computer readable storage medium storing a computer program thereon, wherein the program, when executed by a processor, implements the foregoing cerebral stroke early assessment method and brain region segmentation method.

In the present disclosure, during training, the auto-encoder directs the image segmentation neural network to learn structural features of brain regions, thereby optimizing parameters of the image segmentation neural network. The trained image segmentation neural network 300 segments the preprocessed image, allowing a brain region segmentation image having a higher precision to be obtained.

It should be understood that the brief description above is provided to introduce in a simplified form the technical solutions that will be further described in the Detailed Description of the Embodiments. It is not intended that the brief description above defines the key or essential features claimed of the present disclosure, the scope of which is defined exclusively by the claims. Furthermore, the claimed subject matter is not limited to implementations that solve any disadvantages noted above or in any section of the present disclosure.

These and other features and aspects of the present disclosure will become clearer through the detailed description with reference to the drawings hereinbelow.

### BRIEF DESCRIPTION OF THE DRAWINGS

To obtain a greater understanding of the present disclosure in detail, please refer to the embodiments for a more detailed description of the present disclosure as briefly summarized above. Some embodiments are illustrated in the drawings. In order to facilitate a better understanding, the same symbols have been used as much as possible in the figures to mark the same elements that are common in the various figures. It should be noted, however, that the drawings only illustrate the typical embodiments of the present disclosure and should therefore not be construed as limiting the scope of the present disclosure as the present disclosure may allow other equivalent embodiments. In the figures:

FIG. 1 schematically shows a CT imaging system according to an embodiment of the present disclosure.

FIG. 2 schematically shows a block diagram of a CT imaging system according to an embodiment of the present disclosure.

FIG. 3 schematically shows a block diagram of a cerebral stroke early assessment system according to an embodiment of the present disclosure.

FIG. 4 schematically shows a block diagram of a preprocessing module of a cerebral stroke early assessment system according to an embodiment of the present disclosure.

FIG. 5 schematically shows a structural diagram of a neural network of a brain partitioning module of a cerebral stroke early assessment system according to an embodiment of the present disclosure.

FIG. 6 schematically shows a block flow diagram of a cerebral stroke early assessment method according to an embodiment of the present disclosure.

FIG. 7 schematically shows a block flow diagram of preprocessing of a cerebral stroke early assessment method according to an embodiment of the present disclosure.

FIG. 8 schematically shows an exemplary diagram of brain region segmentation according to an embodiment of the present disclosure.

FIG. 9 schematically shows an exemplary diagram of brain region segmentation according to an embodiment of the present disclosure.

FIG. 10 schematically shows an example of an electronic apparatus for performing a cerebral stroke early assessment method according to an embodiment of the present disclosure.

FIG. 11 schematically shows an example of a cerebral stroke early assessment system according to an embodiment of the present disclosure.

It may be expected that the elements in one embodiment of the present disclosure may be advantageously applied to the other embodiments without further elaboration.

### DETAILED DESCRIPTION

Specific implementations of the present disclosure will be described in the following. It should be noted that during the specific description of the implementations, it is impossible to describe all features of the actual implementations in detail in this description for the sake of brief description. It should be understood that in the actual implementation of any of the implementations, as in the process of any engineering project or design project, a variety of specific decisions are often made in order to achieve the developer's specific objectives and meet system-related or business-related restrictions, which will vary from one implementation to another. Moreover, it can also be understood that although the efforts made in such development process may be complex and lengthy, for those of ordinary skill in the art related to content disclosed in the present disclosure, some changes in design, manufacturing, production or the like based on the technical content disclosed in the present disclosure are only conventional technical means. The content of the present disclosure should not be construed as insufficient.

Unless otherwise defined, the technical or scientific terms used in the claims and the description are as they are usually understood by those of ordinary skill in the art to which the present disclosure pertains. The terms “first,” “second” and similar terms used in the description and claims of the patent application of the present disclosure do not denote any order, quantity or importance, but are merely intended to distinguish between different constituents. “One,” “a(n)” and similar terms are not meant to be limiting, but rather denote the presence of at least one. The term “include,” “comprise” or a similar term is intended to mean that an element or article that appears before “include” or “comprise” encompasses an element or article and equivalent elements that are listed after “include” or “comprise,” and does not exclude other elements or articles. The term “connect,” “connected” or a similar term is not limited to a physical or mechanical connection, and is not limited to a direct or indirect connection.

The cerebral stroke early assessment system and method and the brain region segmentation method described herein may be applied to various medical imaging modalities, including, but not limited to, a computed tomography (CT) apparatus, a magnetic resonance imaging (MRI) apparatus, a positron emission tomography (PET) apparatus, a single photon emission computed tomography (SPECT) apparatus, or any other suitable medical imaging apparatus. The cerebral stroke early assessment system may include the aforementioned medical imaging apparatus, or may include a separate computer apparatus connected to the medical imaging apparatus, and may further include a computer apparatus connected to an Internet cloud. The computer apparatus is connected via the Internet to the medical imaging apparatus or a memory or storage system for storing medical images. The cerebral stroke early assessment method can be implemented independently or jointly by the aforementioned medical imaging apparatus, the computer apparatus connected to the medical imaging apparatus, and the computer apparatus connected to the Internet cloud.

As an example, the present disclosure is described below in conjunction with an X-ray computed tomography (CT) apparatus. As can be appreciated by those skilled in the art, the present disclosure can also be applicable to other medical imaging apparatuses suitable for cerebral stroke early assessment.

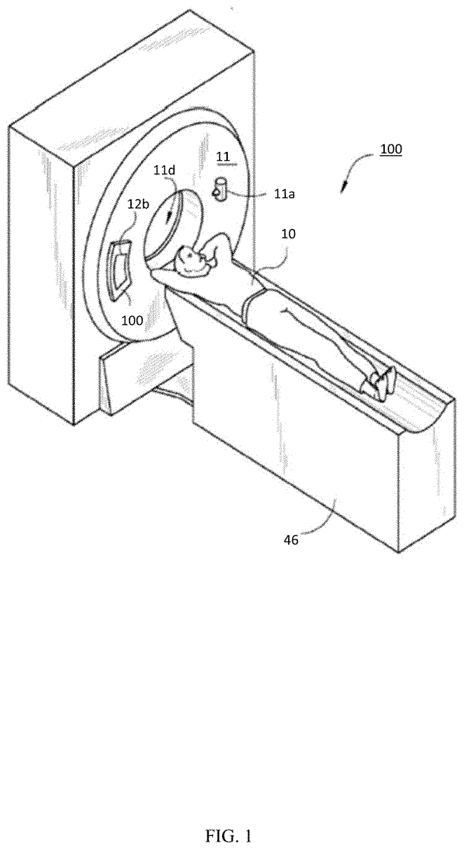

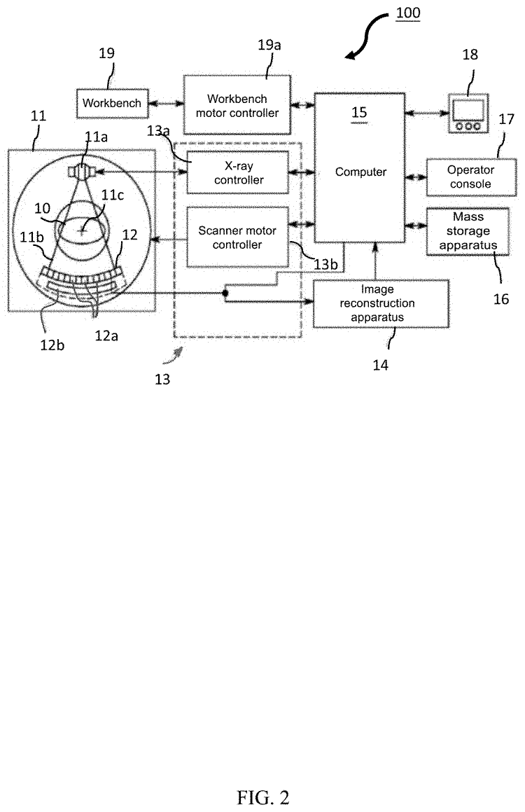

FIG. 1 shows a CT imaging apparatus 100 to which a cerebral stroke early assessment system and method according to an exemplary embodiment of the present disclosure is applicable. FIG. 2 is a schematic block diagram of the example CT imaging apparatus shown in FIG. 1.

Referring to FIG. 1, the CT imaging apparatus 10 is shown as including a scanning gantry 11. The scanning gantry 11 has an X-ray source 11a, and the X-ray source 11a projects an X-ray beam toward a detector assembly or collimator 12 on an opposite side of the scanning gantry 11.

Referring to FIG. 2, the detector assembly 12 includes a plurality of detector units 12a and a data acquisition system (DAS) 12b. The plurality of detector units 12a sense the projected X-ray 11b passing through an object 10.

The DAS 12b converts, according to the sensing of the detector units 12a, collected information into projection data for subsequent processing. During the scanning for acquiring the X-ray projection data, the scanning gantry 11 and components mounted thereon rotate around a rotation center 11c.

The rotation of the scanning gantry 11 and the operation of the X-ray source 11a are controlled by a control mechanism 13 of the CT system 100. The control mechanism 13 includes an X-ray controller 13a that provides power and a timing signal to the X-ray source Ila and a scanner motor controller 13b that controls the rotation speed and position of the scanning gantry 11. An image reconstruction device 14 receives the projection data from the DAS 12b and performs image reconstruction. A reconstructed image is transmitted as an input to a computer 15, and the computer 15 stores the image in a mass storage device 16.

The computer 15 also receives commands and scan parameters from an operator through a console 17, and the console 17 has an operator interface in a certain form, such as a keyboard, a mouse, a voice activated controller, or any other suitable input device. An associated display 18 allows the operator to observe the reconstructed image and other data from the computer 15. The commands and parameters provided by the operator are used by the computer 15 to provide control signals and information to the DAS 12b, the X-ray controller 13a, and the scanning gantry motor controller 13b. In addition, the computer 15 operates a workbench motor controller 19a, which controls a workbench 19 so as to position the object 10 and the scanning gantry 11. In particular, the workbench 19 moves the object 10 in whole or in part to pass through a scanning gantry opening 11d of FIG. 1.

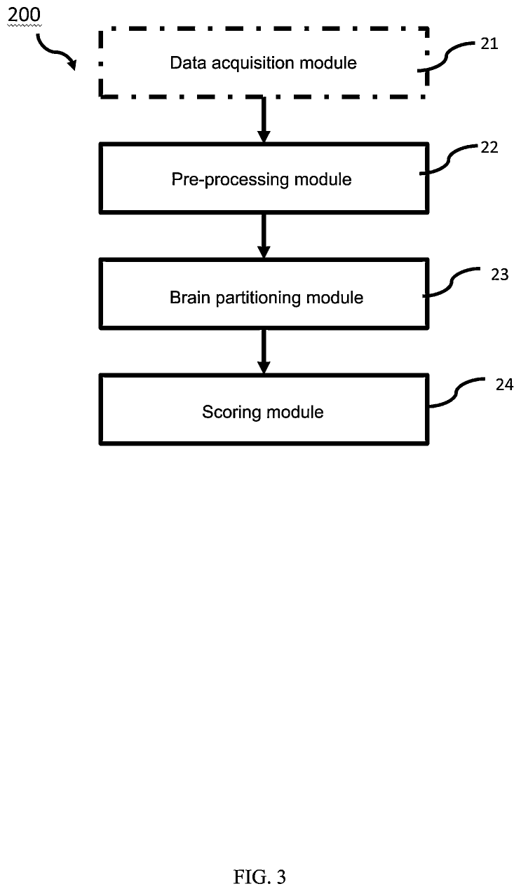

FIG. 3 shows an exemplary block diagram of a cerebral stroke early assessment system 200 according to an embodiment of the present disclosure. The cerebral stroke early assessment system 200 includes a preprocessing module 22, a brain partitioning module 23, a scoring module 24. An acquisition module 21 is configured to acquire a brain medical image data set. The preprocessing module 201 is configured to preprocess the acquired brain medical image data set. The brain partitioning module 23 performs brain region segmentation on the preprocessed brain medical image data set, and the brain partitioning module includes an image segmentation neural network, and the image segmentation neural network is trained with the aid of an auto-encoder. The scoring module 24 is configured to perform scoring on the basis of a brain partition image obtained by the brain partitioning module 23.

Optionally, the cerebral stroke early assessment system 200 may further include a data acquisition module 21, as represented by a dashed box in FIG. 3. The data acquisition module 21 can acquire the brain medical image data set from a brain medical image source, wherein the brain medical image source may include a medical image scanning apparatus such as CT, MRI, PET, PET-CT, PET-MR, SPECT, etc. The brain medical image source may also include a dedicated system for storing brain medical images, such as a picture archiving and communication system (PACS) or a computer cloud storage system. As an example, in cerebral stroke early assessment, the data acquisition module 21 acquires CT plain scan image data or CT perfusion image data. In the case of acquiring the CT perfusion image data, after rapidly intravenous bolus injection of a contrast agent to the brain of a patient, a CT scan is performed on the patient to acquire CT perfusion image data at a plurality of time points at specific intervals. The CT perfusion image data forms the CT brain medical image data set. Preferably, a first stage of CT perfusion image data is acquired. As those skilled in the art can appreciate, the cerebral stroke early assessment system 200 can directly acquire the medical image data from the brain medical image source without the need of the data acquisition module 21.

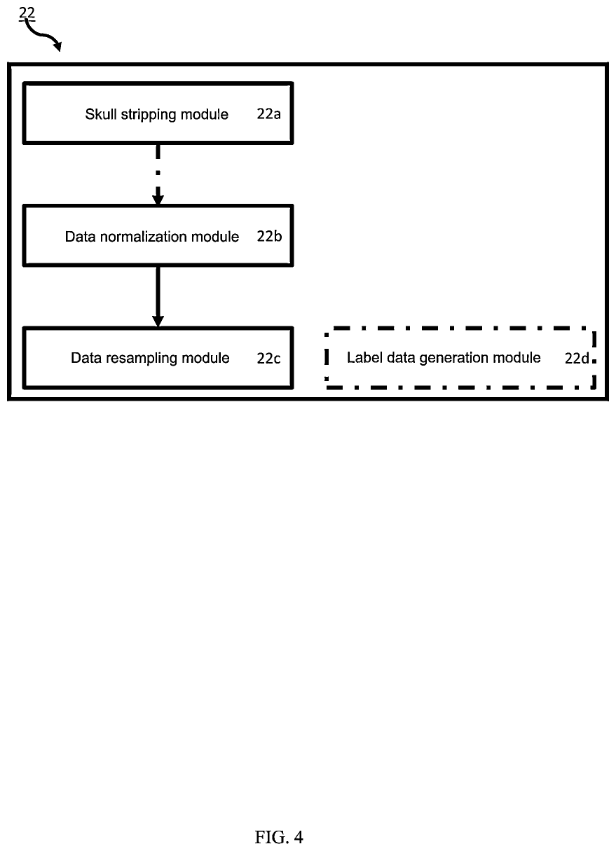

FIG. 4 shows the preprocessing module 22 of the cerebral stroke early assessment system 200 according to an embodiment of the present disclosure. The preprocessing module 22 including a skull stripping module 22a, a data normalization module 22b, and a data resampling module 22c. The preprocessing module 22 preprocesses the acquired brain medical image data set such that the brain medical image data set conforms to particular requirements so as to facilitate input of the preprocessed brain medical image data set into the brain partitioning module for accurate brain region segmentation.

The skull stripping module 22a can strip skull image information in the brain medical image data set, thereby reducing the effect of the skull image information on non-skull image information in subsequent image preprocessing and image segmentation. As an example, the skull stripping module 22a selects the sharpest stage of perfusion image data in the aforementioned acquired CT brain medical image data set, and removes skull image information of the stage of perfusion image data by employing a method such as template matching. In addition, the stage of perfusion image data from which the skull image information has been removed is used as a mask, and then a dot product operation is performed on various other stages of perfusion image data in the brain medical image data set with the mask, so as to obtain a brain medical image data set with the skull image information stripped. Optionally, pixels having an HU value outside the range of [0, 120] in the brain medical image data set may be reset to 0, so as to further optimize skull stripping results. The selection of the sharpest stage of perfusion image data may be based on CT values of pixels in various stages of image data. For example, when the sum of CT values of all pixels in a certain stage of perfusion image data is the highest, or the sum of CT values of a certain proportion of the pixels is the highest, or the average of CT values of all the pixels is the highest, the stage of perfusion image data is selected as the sharpest stage of perfusion image data. As those skilled in the art can appreciate, the operation of the skull stripping module 22a can be automatically performed through a preset procedure without human intervention.

The data normalization module 22b is configured to normalize the acquired brain medical image set. For example, during data normalization, first a mean and a variance of all non-zero image pixel regions in the data set are calculated, then a mean of the overall brain medical image data set is subtracted from each pixel value in the brain medical image data set, and the product is then divided by a variance of the overall brain medical image data set. Normalization is used to control data distribution to a range with a mean of 0 and a standard deviation of 1, to facilitate acceleration of a neural network training process, and reduce the likelihood of a neural network model being trapped in a local optimum.

A data resampling module 22c is configured to resample the data processed by the data normalization module 22b, and is configured to sample data of different dimensions to the same dimension.

As an example, at an image segmentation neural network training phase of the brain partitioning module 23, the preprocessing module 22 further includes a label data generation module 22d represented by a dashed box in FIG. 4 to generate label data that can be manually annotated to a certain amount of brain medical image data. As those skilled in the art can appreciate, at a use phase, the label data generation module 22d may be omitted.

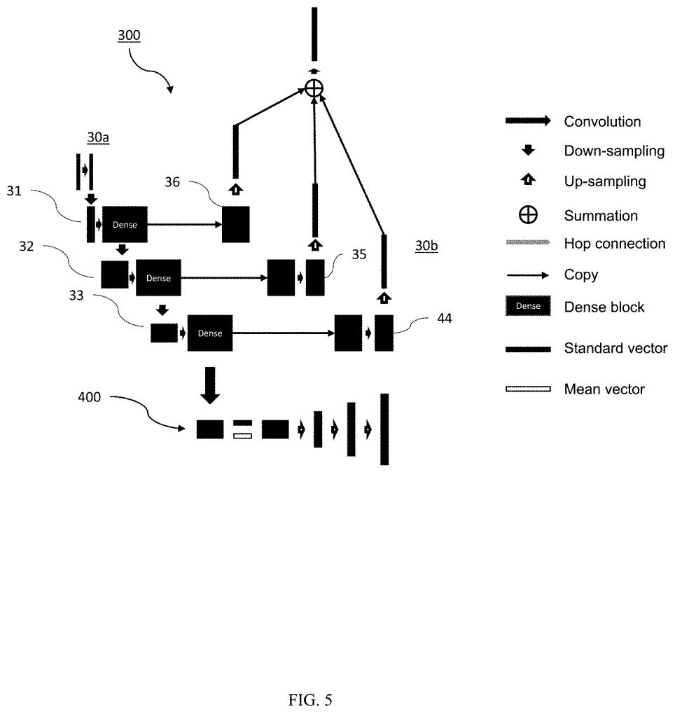

FIG. 5 shows the brain partitioning module 23 according to an embodiment of the present disclosure, configured to perform brain region segmentation on the basis of the preprocessed brain medical image data set. The brain partitioning module 23 includes an image segmentation neural network 300 and an auto-encoder 400, and the image segmentation neural network 300 is trained with the aid of the auto-encoder 400.

As shown in FIG. 5, as an example, the brain partitioning module 23 employs a Dense V-Net neural network 300 as the image segmentation neural network, which includes a down-sampling path (or referred to as a compression path, or a left side path) 30a and an up-sampling path (or referred to as a decompression path, or a right side path) 30b. The down-sampling branch 30a includes three stages of convolutional layers, that is, the down-sampling branch 30a includes a first-stage convolutional layer 31, a second-stage convolutional layer 32, and a third-stage convolutional layer 33, each of which employs a dense block to perform a convolution operation. The dense block includes several convolutional layers in series, and an input of each convolutional layer is formed by stitching together feature maps outputted from all convolutional layers proceeding the layer. An output of the first-stage convolutional layer 31 is down-sampled and then inputted into the second-stage convolutional layer 32, and an output of the second-stage convolutional layer 32 is down-sampled and then inputted into the third-stage convolutional layer 33. In this embodiment, the down-sampling employs a pooling operation (max pool 2×2), which may be a max pooling operation or other suitable pooling operations. The up-sampling path 30b is symmetrical to the down-sampling path 30a, and includes three stages of convolutional layers, that is, includes a fourth-stage convolutional layer 34, a fifth-stage convolutional layer 35, and a sixth-stage convolutional layer 36. An output of the third-stage convolutional layer 33 is directly used as an input of the fourth-stage convolutional layer 34, which is sequentially subjected to a convolution operation and up-sampling and then outputted; the output of the second-stage convolutional layer 35 is directly used as an input of the fifth-stage convolutional layer 35, which is sequentially subjected to a convolution operation and up-sampling and then outputted; and the output of the first-stage convolutional layer 34 is directly used as an input of the sixth-stage convolutional layer 46, which is subjected to a convolution operation and then outputted. The outputs of the three stages of convolutional layers 34, 35, and 46 of the up-sampling path 30b are summed, and the sum passes through a softmax mapping layer to obtain a model predicted segmentation result to achieve brain region segmentation of the brain medical image data set. It can be understood that the brain partitioning module 23 employs the Dense V-Net structure as the image segmentation neural network in this embodiment, and in other implementations, the brain partitioning module 23 may employ a U-Net neural network or a V-Net neural network similar to the Dense V-Net neural network structure.

As shown in FIG. 5, as an example, the auto-encoder 400 employs a variational auto-encoder (VAE), and other kinds of auto-encoders may also be employed in other implementations. The auto-encoder 400 is connected to the down-sampling branch of the image segmentation neural network 300. In this embodiment, the auto-encoder 400 is connected to the third-stage convolutional layer 33 of the down-sampling branch 30a of the image segmentation neural network 300, that is, the third-stage convolutional layer 33 of the down-sampling branch 30a is down-sampled and then inputted into the auto-encoder 400. In other implementations, the auto-encoder 400 may also be connected to the first-stage convolutional layer 31 or the second-stage convolutional layer 32 of the down-sampling branch 30a. The auto-encoder 400 processes the received down-sampled image data with a mean vector and a standard vector, and then outputs a feature image through the three-stage up-sampling layer.

At the training phase, the brain medical image data set and the label data processed by the aforementioned preprocessing module 22 are used for training of the image segmentation neural network 300 and the auto-encoder 400. A loss function for training includes two parts, a KL divergence-based (Kullback-Leibler divergence, also referred to as relative entropy, information divergence) loss function and a segmentation-based Dice coefficient loss function, wherein the variable auto-encoder 400 employs the KL divergence loss function and the image segmentation neural network 300 employs the Dice coefficient loss function. The two losses are summed per iteration as a loss of the overall neural network model of the brain partitioning module 23, as shown by the following equation:

$${L}_{mse \_ loss} + {L}_{dice \_ loss} = {{\sum }^{m}}_{j = 1} {y}_{j \_ true} * log \frac{{y}_{j \_ true}}{{y}_{j \_ predict}} - {\sum }_{i = 1}^{n} \frac{2 T {P}_{i}}{2 T {P}_{i} + F {N}_{i} + F {P}_{i}}$$

y_{j_predict} represents the jth pixel data of a recovered image predicted by the auto-encoder model, y_{j_true} true represents the jth pixel data of an input image, n represents a predicted ASPECTS category, and TPᵢ, FNᵢ, and FPᵢ represent a true positive rate, a false negative rate, and a false positive rate in a category i segmentation result, respectively.

It can be understood that in the implementation of the embodiment of the present disclosure, at the training phase, training of the image segmentation neural network 300 and the auto-encoder 400 is a multitasking parallel learning, and there is an interaction between training results of the two networks. The segmentation precision of the image segmentation neural network 300 depends on brain structural features learned by the network. That is, if the brain structural features accurately represent structural detail information of the brain, the image segmentation neural network 300 has high image segmentation precision, and can be applied to image data derived from different scanning apparatuses, i.e., has better generalization performance. The auto-encoder 400 reconstructs an original brain image with the feature layer information down-sampled by the third-stage convolutional layer 33, and the auto-encoder 400 can learn to obtain structural information of the brain image. As mentioned above, the sum of the two losses in the training isused as the loss of the overall neural network model. By merging the loss functions, shared parameters of the image segmentation neural network 400 can be optimized, and said part of shared parameters can express the brain structure information. That is, in the training process, the image segmentation neural network 300 is trained with the aid of the auto-encoder 400, so that parameters of the image segmentation neural network 300 can be optimized and the precision of image segmentation can be improved.

As shown in FIG. 3, the cerebral stroke early assessment system 200 further includes a scoring module 24. The scoring module 24 scores the severity of a cerebral stroke according to an ASPECTS scoring rule on the basis of the brain partition image acquired via processing by the brain partitioning module 23.

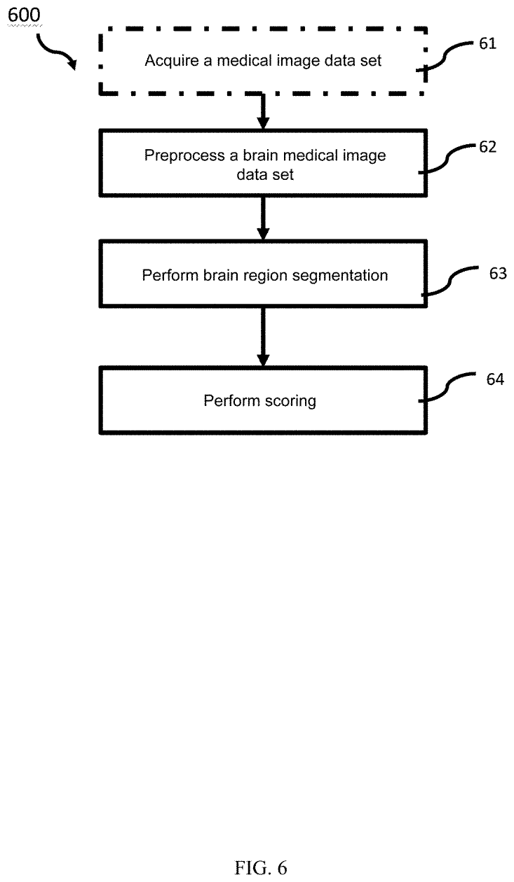

FIG. 6 shows an example block diagram of a cerebral stroke early assessment method 600 according to embodiments of the present disclosure. The cerebral stroke early assessment method 600 includes a brain medical image data set preprocessing step 62, a brain region segmentation step 63, and an ASPECTS scoring step 64.

Optionally, the cerebral stroke early assessment method 600 may further include a brain medical image data set acquisition step 61 as represented by a dashed box in FIG. 6. In step 61, the brain medical image data set may be acquired from a brain medical image source, wherein the brain medical image source may include a medical image scanning apparatus such as CT, MRI, PET, PET-CT, PET-MR, SPECT, etc. The brain medical image source may also include a dedicated system for storing brain medical images, e.g., a picture archiving and communication system (PACS) or a computer cloud storage system. As an example, the brain medical image data set acquired in step 61 includes CT brain plain scan data or CT perfusion image data. In the case of acquiring the CT perfusion image data, after rapidly intravenous bolus injection of a contrast agent to the brain of a patient, a CT scan is performed on the patient to acquire CT perfusion image data at a plurality of time points at specific intervals. The CT perfusion image data forms the CT brain medical image data set. Preferably, the first stage of CT perfusion image data is acquired. As those skilled in the art can appreciate, the cerebral stroke early assessment method 600 can acquire the medical image data directly from the brain medical image source without the need of the brain medical image data set acquisition step 61.

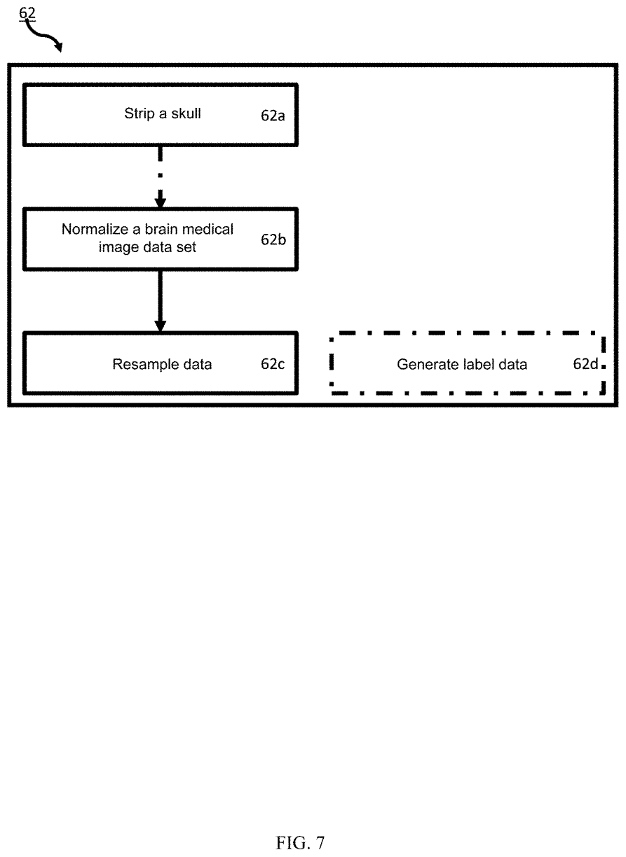

FIG. 7 shows the brain medical image data set preprocessing step 62. Step 62 includes a skull stripping step 62a, a brain medical image data set normalization step 62b, and a data resampling step 62c. The preprocessed image data meets specific requirements, so as to facilitate further acquisition of proper brain region segmentation.

In step 62a, the skull is stripped. Skull image information in the CT brain medical image data set can be stripped, thereby reducing the effect of the skull image information on non-skull image information in subsequent image preprocessing and image segmentation. As an example, in step 62a, the sharpest stage of brain medical image data in the aforementioned brain medical image data set is selected, and the skull in the stage of brain medical image data is removed by employing a method such as template matching. Meanwhile, the stage of brain medical image data from which the skull image information has been removed is used as a mask, and then a dot product operation is performed on various other stages of brain medical image data with the mask, so as to obtain a multi-stage brain medical image data set with the skull image information stripped. Optionally, pixels having an HU value outside the range of [0, 120] in the brain medical image data set may be further reset to 0, so as to further optimize skull stripping results. The selection of the sharpest stage of brain medical image data set described above may be based on CT values of pixels in various stages of image data. For example, when the sum of CT values of all pixels in a certain stage of perfusion image data is the highest, or the sum of CT values of a certain proportion of the pixels is the highest, or the average of CT values of all the pixels is the highest, the stage of CT brain medical image data is selected as the sharpest stage of brain medical image data. As those skilled in the art can appreciate, the skull stripping step can be automatically performed via a preset procedure without human intervention.

In step 62b, the brain medical image data set is normalized. As an example, first a mean and a variance of all non-zero image pixel regions in the data set is calculated, then a mean of the overall brain medical image data set is subtracted from each pixel value in the brain medical image data set, and the product is then divided by a variance of the overall brain medical image data set. Normalization is used to control data distribution to a range with a mean of 0 and a standard deviation of 1, to facilitate the acceleration of a neural network training process, and reduce the likelihood of a neural network model being trapped in a local optimum.

In step 62c, data resampling is performed, which will be used to resample the brain medical image data set processed by the data normalization module 22b to sample data of different dimensions to the same dimension.

As an example, at an image segmentation neural network training phase used in the brain region segmentation step 63, the brain medical image data set preprocessing step 62 may further include a label data generation step 62d represented by a dashed box in FIG. 7 to generate label data. The label data can be manually annotated to a certain amount of brain medical image data. As those skilled in the art can appreciate, at a use phase, the label data generation step 62d may be omitted.

In step 63, the brain region segmentation uses the image segmentation neural network 300 and the auto-encoder 400 as shown in FIG. 5. At the training phase, the aforementioned brain medical image data set generated by preprocessing and the label data are used for training of the image segmentation neural network 300 and the auto-encoder 400. At the actual use phase, the trained image segmentation neural network 300 performs image segmentation on the preprocessed brain medical image data set.

In step 64, the scoring step 64 scores the severity of a cerebral stroke according to an ASPECTS scoring rule on the basis of the brain partition image acquired in the brain region segmentation step 63.

It can be understood that in the present disclosure, the auto-encoder 400 will direct the image segmentation neural network 300 to learn structural features of brain regions in the training process, thereby optimizing parameters of the image segmentation neural network 300. At the use phase, the trained image segmentation neural network 300 segments the preprocessed image to obtain a brain region segmentation image with higher precision, and further obtain an accurate brain stroke score.

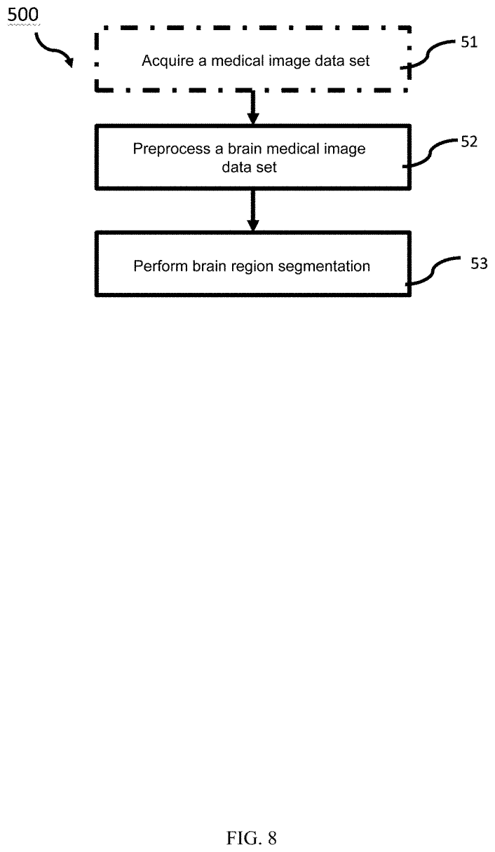

FIG. 8 shows a brain region segmentation method 500 for a brain medical image according to an embodiment of the present disclosure, comprising an acquired brain medical image preprocessing step 52 and a brain region segmentation step 53. Optionally, the brain region segmentation method 500 includes a brain medical image acquisition step 51 as represented by a dashed box in FIG. 8. The brain medical image acquisition step 51 is the same as the brain medical image data set acquisition step 61 of the cerebral stroke early assessment method 600 described above. The acquired brain medical image preprocessing step 52 is the same as the brain medical image data set preprocessing step 62 of the cerebral stroke early assessment method 600 described above. The brain region segmentation step 53 uses an image segmentation neural network to perform brain region segmentation on the preprocessed brain medical image. The image segmentation neural network is trained with the aid of an auto-encoder. The brain region segmentation uses an image segmentation neural network 300 and an auto-encoder 400 as shown in FIG. 5. At a training phase, the aforementioned brain medical image data set generated by preprocessing and label data are used for training of the image segmentation neural network 300 and the auto-encoder 400. At an actual use phase, the trained image segmentation neural network 300 performs image segmentation on the preprocessed brain medical image data set.

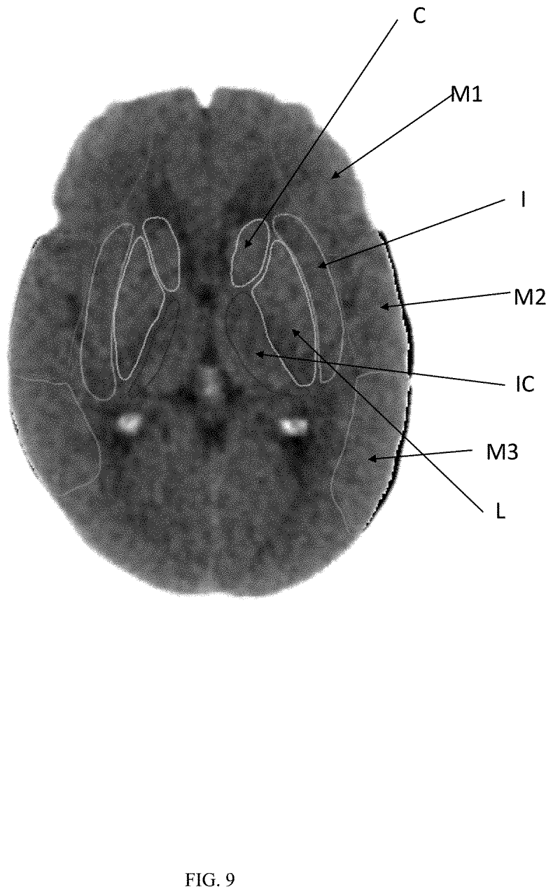

FIG. 9 shows an exemplary diagram of brain region segmentation results of the cerebral stroke early assessment method 600 and the brain region segmentation method 500 for a brain medical image according to an embodiment of the present disclosure.

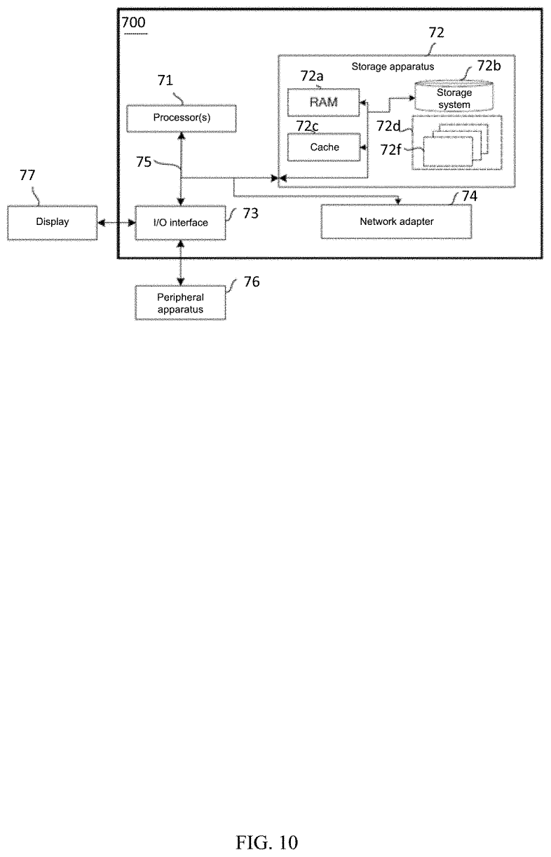

FIG. 10 shows an example of an electronic apparatus 700 for performing a cerebral stroke early assessment method according to an embodiment of the present disclosure. The electronic apparatus 700 includes: one or a plurality of processors 71; and a storage device 72, configured to store one or a plurality of programs, where when the one or plurality of programs are executed by the one or plurality of processors 71, the one or plurality of processors 71 are caused to implement the method described herein. The processor is, for example, a digital signal processor (DSP), a microcontroller, an application-specific integrated circuit (ASIC), or a microprocessor.

The electronic apparatus 700 shown in FIG. 10 is only an example, and should not bring any limitation to the function and application scope of the embodiment of the present disclosure.

As shown in FIG. 10, the electronic apparatus 700 is represented in the form of a general-purpose computing device. The components of the electronic apparatus 700 may include, but is not limited to, one or a plurality of processors 71, a storage device 72, and a bus 75 connecting different system components (including the storage device 72 and the processor 71).

The bus 75 represents one or a plurality of types of bus structures, including a memory bus or a memory controller, a peripheral bus, an accelerated graphics port, a processor, or a local bus using any bus structure in the plurality of bus structures. For example, these architectures include, but are not limited to, an Industrial Standard Architecture (ISA) bus, a Micro Channel Architecture (MAC) bus, an enhanced ISA bus, a Video Electronics Standards Association (VESA) local bus, and a Peripheral Component Interconnect (PCI) bus.

The electronic apparatus 700 typically includes a variety of computer system readable media. These media may be any available medium that can be accessed by the electronic apparatus 700, including volatile and non-volatile media as well as removable and non-removable media.

The storage apparatus 72 may include a computer system readable medium in the form of a volatile memory, for example, a random access memory (RAM) 72a and/or a cache memory 72c. The electronic apparatus 700 may further include other removable/non-removable, and volatile/non-volatile computer system storage media. Only as an example, a storage system 72b may be configured to read/write a non-removable, non-volatile magnetic medium (not shown in FIG. 10, often referred to as a “hard disk drive”). Although not shown in FIG. 10, a magnetic disk drive configured to read/write a removable non-volatile magnetic disk (for example, a “floppy disk”) and an optical disk drive configured to read/write a removable non-volatile optical disk (for example, a CD-ROM, a DVD-ROM, or other optical media) may be provided. In these cases, each drive may be connected to the bus 75 via one or a plurality of data medium interfaces. The storage device 72 may include at least one program product which has a group of program modules (for example, at least one program module) configured to perform the functions of the embodiments of the present disclosure.

A program/utility tool 72d having a group (at least one) of program modules 72f may be stored in, for example, the storage apparatus 72. Such a program module 72f includes, but is not limited to, an operating system, one or a plurality of application programs, other program modules, and program data, and each of these examples or a certain combination thereof may include the implementation of a network environment. The program module 72f typically performs the function and/or method in any embodiment described in the present disclosure.

The electronic apparatus 700 may also communicate with one or a plurality of peripheral devices 76 (such as a keyboard, a pointing device, and a display 77), and may further communicate with one or a plurality of devices that enable a user to interact with the electronic apparatus 700, and/or communicate with any device (such as a network card and a modem) that enables the electronic apparatus 700 to communicate with one or a plurality of other computing devices. Such communication may be carried out via an input/output (I/O) interface 73. In addition, the electronic apparatus 700 may also communicate with one or a plurality of networks (for example, a local area network (LAN), a wide area network (WAN), and/or a public network, such as the Internet) via a network adapter 74. As shown in FIG. 10, the network adapter 74 communicates with other modules of the electronic apparatus 700 through the bus 75. It should be understood that although not shown in the drawing, other hardware and/or software modules, including, but not limited to: microcode, device drivers, redundant processing units, external disk drive arrays, RAID systems, tape drives, data backup storage system, and the like may be used in conjunction with the electronic apparatus 700.

The processor 71 executes various functional applications and data processing by running programs stored in the storage apparatus 72.

According to an embodiment of the present disclosure, a computer readable medium is further provided. The computer readable medium has instructions thereon, and when executed by a processor, the instructions cause the processor to perform the steps of the method of the present disclosure. The computer-readable medium may include, but is not limited to, a non-transitory, tangible arrangement of an article manufactured or formed by a machine or apparatus, including a storage medium such as the following: a hard disk; any other type of disk including a floppy disk, an optical disk, a compact disk read-only memory (CD-ROM), a compact disk rewritable (CD-RW), and a magneto-optical disk; a semiconductor device such as a read-only memory (ROM), a random access memory (RAM) such as a dynamic random access memory (DRAM) and a static random access memory (SRAM), an erasable programmable read-only memory (EPROM), a flash memory, and an electrically erasable programmable read-only memory (EEPROM); a phase change memory (PCM); a magnetic or optical card; or any other type of medium suitable for storing electronic instructions. The computer-readable medium may be installed in a CT device, or may be installed in a separate control device or computer that remotely controls the CT device.

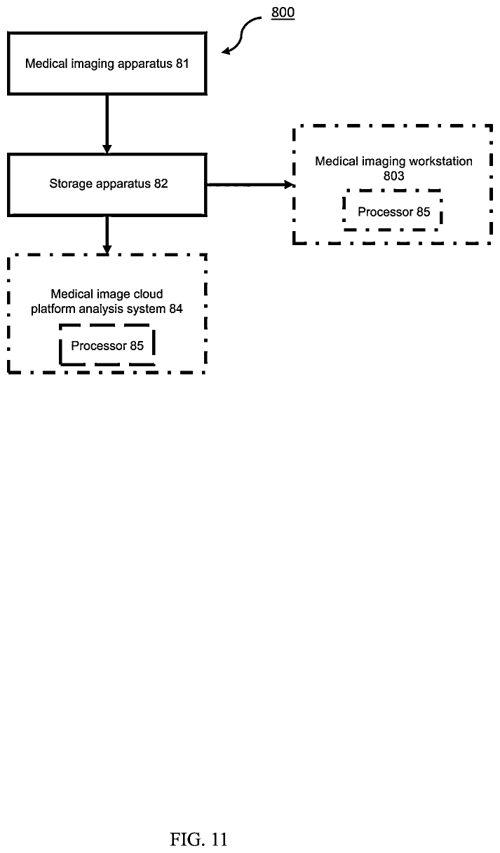

FIG. 11 shows a block diagram of an example cerebral stroke early assessment system 800 according to an embodiment of the present disclosure. Referring to FIG. 11, the cerebral stroke early assessment system 800 may include a medical imaging apparatus 81 configured to perform imaging scanning to generate a medical image, a storage apparatus 82 configured to store the medical image, and a medical imaging workstation 83 or a medical image cloud platform analysis system 84 communicatively connected to the storage apparatus 82, and including a processor 85. The processor 85 can be used to perform the cerebral stroke early assessment method of the present disclosure described above.

The medical imaging device 81 can be a CT apparatus, an MRI apparatus, a PET apparatus, a SPECT apparatus, or any other suitable imaging apparatus. The storage apparatus 82 may be located in the medical imaging apparatus 81, a server external to the medical imaging apparatus 81, an independent medical image storage system (such as a PACS), and/or a remote cloud storage system. The medical imaging workstation 83 may be disposed locally at the medical imaging apparatus 81, that is, the medical imaging workstation 83 being disposed adjacent to the medical imaging apparatus 81, and the two may be co-located in a scanning room, a medical imaging department, or the same hospital. The medical image cloud platform analysis system 84 may be located away from the medical imaging apparatus 81, for example, arranged at the cloud in communication with the medical imaging apparatus 81. As an example, after a medical institution completes an imaging scan using the medical imaging apparatus 81, data obtained by the scanning is stored in the storage apparatus 82. The medical imaging workstation 83 may directly obtain the data obtained by the scanning, and perform subsequent analysis by using the method of the present disclosure via its processor. As another example, the medical image cloud platform analysis system 84 may read the medical image in the storage apparatus 82 via remote communication to provide “software as a service (SAAS).” The SAAS may exist between hospitals, between a hospital and an imaging center, or between a hospital and a third-party online diagnosis and treatment service platform.

The technology described in the present disclosure may be implemented at least in part through hardware, software, firmware, or any combination thereof. For example, aspects of the technology may be implemented through one or more microprocessors, digital signal processors (DSP), application-specific integrated circuits (ASIC), field programmable gate arrays (FPGA), or any other equivalent integrated or separate logic circuits, and any combination of such parts embodied in a programmer (such as a doctor or patient programmer, stimulator, or the other apparatuses). The term “processor”, “processing circuit”, “controller” or “control module” may generally refer to any of the above noted logic circuits (either alone or in combination with other logic circuits), or any other equivalent circuits (either alone or in combination with other digital or analog circuits).

Some illustrative embodiments of the present disclosure have been described above. However, it should be understood that various modifications can be made to the exemplary embodiments described above without departing from the spirit and scope of the present disclosure. For example, an appropriate result can be achieved if the described techniques are performed in a different order and/or if the components of the described system, architecture, apparatus, or circuit are combined in other manners and/or replaced or supplemented with additional components or equivalents thereof; accordingly, the modified other embodiments also fall within the protection scope of the claims.

What is claimed is:

1. A medical image-based cerebral stroke early assessment system, comprising:
a preprocessing module, configured to preprocess an acquired brain medical image;
a brain partitioning module, configured to perform brain region segmentation on the preprocessed brain medical image, the brain partitioning module comprising an image segmentation neural network, the image segmentation neural network being trained with the aid of an auto-encoder; and
a scoring module, configured to perform scoring on the basis of a brain partition image obtained by the brain partitioning module.

2. The cerebral stroke early assessment system according to claim 1, wherein the image segmentation neural network comprises a Dense V-Net neural network, a U-Net neural network, or a V-Net neural network.

3. The cerebral stroke early assessment system according to claim 2, wherein the auto-encoder comprises a variational auto-encoder.

4. The cerebral stroke early assessment system according to claim 3, wherein the auto-encoder is connected to a down-sampling branch of the image segmentation neural network.

5. The cerebral stroke early assessment system according to claim 4, wherein a loss function trained by the image segmentation neural network comprises a KL divergence loss function and a Dice coefficient loss function, wherein the KL divergence loss function corresponds to a loss function of the auto-encoder, and the Dice coefficient loss function corresponds to a loss function of the image segmentation neural network.

6. A medical image-based cerebral stroke early assessment method, comprising:
preprocessing an acquired brain medical image;
performing brain region segmentation on the preprocessed brain medical image, the brain region segmentation using an image segmentation neural network and the image segmentation neural network being trained with the aid of an auto-encoder; and
performing scoring on the basis of a brain partition image obtained by the brain partitioning module.

7. The cerebral stroke early assessment method according to claim 6, wherein the image segmentation neural network comprises a Dense V-Net neural network, a U-Net neural network, or a V-Net neural network.

8. The cerebral stroke early assessment method according to claim 7, wherein the auto-encoder comprises a variational auto-encoder.

9. The cerebral stroke early assessment method according to claim 8, wherein the auto-encoder is connected to a down-sampling branch of the image segmentation neural network.

10. The cerebral stroke early assessment method according to claim 9, wherein a loss function trained by the image segmentation neural network comprises a KL divergence loss function and a Dice coefficient loss function, wherein the KL divergence loss function corresponds to a loss function of the auto-encoder, and the Dice coefficient loss function corresponds to a loss function of the image segmentation neural network.

11. A brain region segmentation method for a brain medical image, comprising:
preprocessing an acquired brain medical image; and
performing brain region segmentation on the preprocessed brain medical image by using an image segmentation neural network, the image segmentation neural network being trained with the aid of an auto-encoder.

12. The brain medical image partitioning method according to claim 11, wherein the image segmentation neural network comprises a Dense V-Net neural network, a U-Net neural network, or a V-Net neural network.

13. The brain medical image partitioning method according to claim 12, wherein the auto-encoder comprises a variational auto-encoder.

14. The brain medical image partitioning method according to claim 13, wherein the auto-encoder is connected to a down-sampling branch of the image segmentation neural network.

15. The brain medical image partitioning method according to claim 14, wherein a loss function trained by the image segmentation neural network comprises a KL divergence loss function and a Dice coefficient loss function, wherein the KL divergence corresponds to a loss function of the auto-encoder, and the Dice coefficient loss function corresponds to a loss function of the image segmentation neural network.
